(19) United States
(12) Patent Application Publication
(10) Pub. No.: US 2024/0277980 A1
(43) Pub. Date: Aug. 22, 2024

(54) MEDICAL DEVICE HAVING SUPPORT MEMBER

(71) Applicant: ABBOTT CARDIOVASCULAR SYSTEMS INC., Santa Clara, CA (US)

(72) Inventors: Shane J. O'Neill, Waterford (IE); Diarmuid J. Wall, Clonmel (IE); Joseph S. Keane, Campile (IE); Rok Kordez, Clonmel (IE)

(21) Appl. No.: 18/437,043

(22) Filed: Feb. 8, 2024

Related U.S. Application Data
(60) Provisional application No. 63/447,022, filed on Feb. 20, 2023.

(51) Int. Cl. A61M 25/10; A61M 25/00

(52) U.S. Cl. CPC A61M 25/104; A61M 25/0021; A61M 25/005; A61M 25/0054; A61M 25/1002; A61M 25/1027

(57) ABSTRACT

A catheter includes a midshaft having a proximal midshaft end and a distal midshaft end. An inner shaft extends distally through the midshaft to a distal inner shaft end. A balloon has a proximal balloon shoulder coupled to the distal midshaft end and a distal balloon shoulder coupled to the inner shaft. A proximal shaft is coupled to the proximal midshaft end. The proximal shaft includes a support member that extends through the balloon at least to the distal balloon shoulder.

This application claims the benefit of priority of U.S. Provisional Patent Application No. 63/447,022, filed on Feb. 20, 2023, titled “MEDICAL DEVICE HAVING SKIVED SHAFT,” which is incorporated herein by reference in its entirety to provide continuity of disclosure.

### FIELD

Various embodiments of the disclosure relate to medical devices, and more specifically to catheter-based interventional medical devices such as balloon dilatation catheters and stent delivery systems that include a skived shaft.

### BACKGROUND

A conventional percutaneous transluminal angioplasty (PTA) is a minimally invasive procedure in which a blocked (or partially blocked) artery, such as a coronary artery of a patient's heart muscle, is opened in order to improve blood flow to the heart muscle. First, a guidewire is advanced into the patient's coronary artery until the distal end of the guidewire crosses a lesion to be dilated. A dilatation catheter, having an inflatable balloon on the distal portion thereof, is advanced into the patient's coronary anatomy over the previously introduced guidewire until the balloon of the dilatation catheter is properly positioned across the lesion. Once properly positioned, the dilatation balloon is inflated with inflation fluid one or more times to a predetermined size at relatively high pressures so that the arterial wall expands to open up the vascular passageway. After the balloon is deflated, blood resumes through the dilated artery and the dilatation catheter and the guidewire can be removed therefrom.

### SUMMARY

Critical Limb Ischemia (CLI) is a severe blockage in a patient's lower extremities (e.g., in a leg, ankle, foot), which as a result may reduce (or stop) blood-flow to the extremities. In particular, CLI may be the result of narrowing of arteries (or arteries that are calcified) over time due to a buildup of fatty deposits called plaque. Patients with CLI often have diabetic foot ulcers or gangrene, which results in severe pain, and are at elevated risk of amputation as a result of these arterial blockages, such as those below the ankle. A conventional balloon catheter used for a PTA may be unable to effectively access and efficiently dilate a lower extremity artery (e.g., the peroneal artery, the dorsalis pedis artery, plantar arteries, etc.) to address a patient's CLI. Unlike coronary arteries for which most (if not all) conventional catheters are designed, lower extremity arteries may be more tortuous and thinner. As a result, conventional catheters may not be flexible enough and have a thin enough profile to be able to effectively navigate through a lower extremity artery for an operator to perform a PTA at a location where a blockage is located. Therefore, there is a need for a catheter-based interventional medical device that is capable of navigating through lower extremity arteries to perform a PTA procedure.

The present disclosure provides a medical device, such as a balloon dilatation catheter that is designed to treat stenotic arterial occlusions in lower extremity arteries. The device includes a midshaft having a proximal midshaft end and a distal midshaft end, and includes an inner shaft extending distally through the midshaft to a distal inner shaft end. The device also includes a balloon having a proximal balloon shoulder coupled to the distal midshaft end and a distal balloon shoulder coupled to the inner shaft. The device includes a proximal shaft coupled to the proximal midshaft end. The proximal shaft includes a support member that extends through the balloon at least to the distal balloon shoulder. Thus, unlike conventional balloon catheters, the device of the present disclosure includes a support member that extends through the balloon to the distal balloon shoulder, which provides the device with extra support, thereby providing better trackability through tortuous anatomy and calcified arteries in lower extremities during a PTA procedure.

In one embodiment, a catheter is provided. The catheter includes a midshaft, an inner shaft, a balloon, and a proximal shaft. The midshaft has a distal midshaft end. The inner shaft extends distally through the midshaft to a distal inner shaft end. The balloon has a proximal balloon shoulder coupled to the distal midshaft end and a distal balloon shoulder coupled to the inner shaft. The proximal shaft includes a support member that extends through the balloon at least to the distal balloon shoulder.

In one embodiment, a catheter is provided. The catheter includes a proximal shaft, a midshaft, a balloon, an inner shaft, and an intermediate shaft. The midshaft is coupled to the proximal shaft and includes a midshaft lumen. The balloon includes a balloon interior that is in fluid communication with the midshaft lumen. The balloon includes a proximal balloon shoulder and a distal balloon shoulder. The inner shaft extends distally through the midshaft lumen from a proximal inner shaft end to a distal inner shaft end. The inner shaft is coupled to the distal balloon shoulder. The intermediate shaft includes a shaft wall that at least partially surrounds an intermediate shaft lumen through which the midshaft lumen fluidly communicates with the balloon interior. The intermediate shaft has a plurality of cuts in a cut pattern, and is coupled to the proximal shaft and to the proximal balloon shoulder of the balloon. At least a portion of the inner shaft extends through the intermediate shaft lumen.

In one embodiment, a method of manufacturing a catheter is provided. The method includes cutting a proximal shaft at an angle along a longitudinal axis that runs through the proximal shaft to form a skive section that has an arc-shaped cross section. The method includes coupling a distal end of a midshaft to a proximal balloon shoulder of a balloon. A midshaft lumen is in fluid communication with a balloon interior of the balloon. The method includes coupling a distal end of the proximal shaft to a distal balloon shoulder of the balloon. The skive section is at least disposed within the midshaft lumen of the midshaft. The method includes coupling an inner shaft to the distal balloon shoulder and coupling the inner shaft to the midshaft.

The above summary does not include an exhaustive list of all embodiments of the disclosure. It is contemplated that the disclosure includes all systems and methods that can be practiced from all suitable combinations of the various embodiments summarized above, as well as those disclosed in the Detailed Description below and particularly pointed out in the claims. Such combinations may have particular advantages not specifically recited in the above summary.

### BRIEF DESCRIPTION OF THE DRAWINGS

The embodiments are illustrated by way of example and not by way of limitation in the figures of the accompanying drawings in which like references indicate similar elements. It should be noted that references to “an” or “one” embodiment of this disclosure are not necessarily to the same embodiment, and they mean at least one. Also, in the interest of conciseness and reducing the total number of figures, a given figure may be used to illustrate the features of more than one embodiment, and not all elements in the figure may be required for a given embodiment.

FIG. 1 shows a side view of a medical device, in accordance with an embodiment.

FIG. 2 shows a side perspective view of a proximal shaft of the medical device, in accordance with an embodiment.

FIGS. 3-5 show cross-sectional views of the medical device, in accordance with an embodiment.

FIG. 6 shows a side view of the medical device, in accordance with an embodiment.

FIG. 7 shows another cross-sectional view of the medical device, in accordance with an embodiment.

FIG. 8 shows a side view of the medical device, in accordance with an embodiment.

FIGS. 9 and 10 each show a cut pattern according to an embodiment.

FIG. 11 is a flowchart of a method of manufacturing a medical device, in accordance with an embodiment.

FIG. 12 shows a side view of the medical device, in accordance with an embodiment.

FIG. 13 shows another cross-sectional view of the medical device, in accordance with an embodiment.

### DETAILED DESCRIPTION

Several embodiments of the disclosure with reference to the appended drawings are now explained. Whenever the shapes, relative positions and other embodiments of the parts described in the embodiments are not explicitly defined, the scope of the disclosure is not limited only to the parts shown, which are meant merely for the purpose of illustration. Also, while numerous details are set forth, it is understood that some embodiments of the disclosure may be practiced without these details. In other instances, structures and techniques have not been shown in detail so as not to obscure the understanding of this description. Furthermore, unless the meaning is clearly to the contrary, all ranges set forth herein are deemed to be inclusive of the endpoints. In addition, the terms “over,” “to,” and “on” as used herein may refer to a relative position of one feature with respect to other features. One feature “over” or “on” another feature or bonded “to” another feature may be directly in contact with the other feature or may have one or more intervening layers. In addition, the use of relative terms throughout the description, such as “top,” “above or “upper” and “bottom,” “under” or “lower” may denote a relative position or direction. For example, a “top edge,” “top end” or “top side” may be directed in a first axial direction and a “bottom edge,” “bottom end” or “bottom side” may be directed in a second direction opposite to the first axial direction.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “distal to” may indicate a first direction away from a reference point. Similarly, “proximal to” may indicate a location in a second direction away from the reference point and opposite to the first direction. Such terms are provided to establish relative frames of reference, however, and are not intended to limit the use or orientation of a medical device to a specific configuration described in the various embodiments herein.

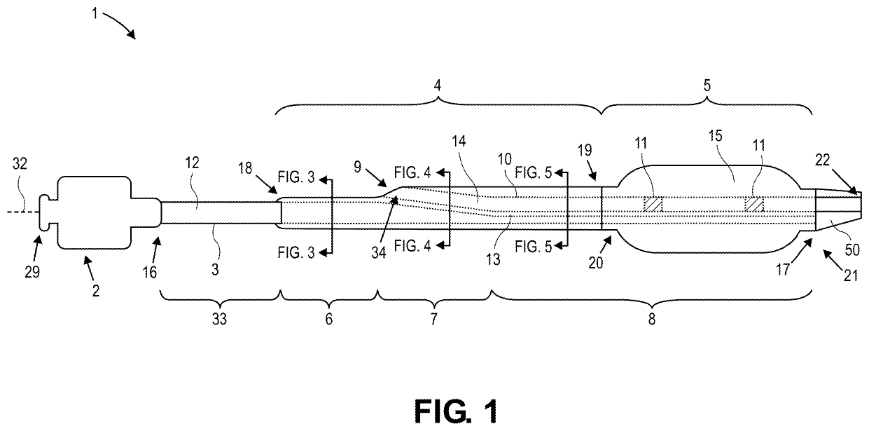

FIG. 1 shows a side view of a medical device 1, such as a balloon dilatation catheter or a stent delivery system, which hereafter may be referred to as “catheter” or “device”, in accordance with an embodiment. The device 1 includes an adapter 2, a proximal shaft 3, an inner shaft 10, a midshaft 4, and a balloon 5. In some embodiments, the device may include less or more components. For example, the device may include a stent that is mounted on the balloon 5 (not shown), which may be used for a stenting procedure (e.g., in a lower extremity artery) in which dilatation is performed at a location with arterial occlusion and the stent is positioned at or near the location. In one embodiment, the proximal shaft 3 and midshaft 4 may form or be a part of a catheter shaft of the device that couples the adapter 2 to the balloon 5. As shown, the device includes a longitudinal axis 32 extending centrally through at least one of the components of the device. For instance, the axis 32 runs centrally through at least a portion of the proximal shaft 3, the midshaft 4, the inner shaft 10, and/or the balloon 5. In another embodiment, the axis may not be a central axis to at least a portion of one of the components, such as the midshaft 4 and/or the inner shaft 10.

The proximal shaft 3 extends from the adapter 2 to at least the balloon 5. In particular, the proximal shaft 3 includes a proximal end 16 that is coupled to (e.g., a distal end of) the adapter 2, and includes a distal end 17 that is coupled to the balloon 5. In one embodiment, the proximal shaft 3 may extend along the longitudinal axis 32 beyond (and out of) the balloon. The proximal shaft 3 includes an outer portion 33, a proximal portion 6, a distal (or skive) portion 7, and a support member 8. As shown, the proximal shaft 3 extends distally from the adapter 2 into the midshaft 4 and the balloon 5. In particular, the proximal portion 6 and the distal portion 7 are disposed (e.g., entirely) within a midshaft lumen 13 of the midshaft 4, where the proximal portion extends proximally within the midshaft lumen 13 from a proximal midshaft end 18 of the midshaft 4, and the distal portion 7 extends distally from the proximal portion 6. In addition, the support member 8, which is coupled to, e.g., by attachment to (e.g., via an adhesive and/or thermal bond) or by being integrally formed with, the distal portion 7 extends distally into (or through) the midshaft lumen 13 and is at least partially disposed within the midshaft lumen 13 and/or (a balloon interior 15 of) the balloon 5. The outer portion 33, however, which extends from the proximal end 16 at the adapter 2 to a proximal midshaft end 18 of the midshaft 4 may not be disposed within the midshaft 4, but may instead be within (or exposed to) the ambient environment. In one embodiment, at least some of the elements of the proximal shaft 3, such as the proximal portion 6, the distal portion 7, and/or the support member 8, may be one unit. For instance, each of the elements may be formed or molded from one or more materials into one integrated structure. In another embodiment, at least some of the portions may be attached or bonded, through an adhesive or a thermal bond, to one another.

As shown, the support member 8 of the proximal shaft 3 extends through a portion of the midshaft 4 and extends through the balloon 5 at least to the distal balloon shoulder 21, to which the member is attached. In particular, the proximal shaft 3 extends distally within the midshaft lumen 13 and within the balloon interior 15 to a distal end 17 at or distal to the distal balloon shoulder 21. For example, the distal end 17 may be coupled to the distal balloon 21, or the distal end 17 may extend out of the distal balloon shoulder 21 into the environment. In another embodiment, the distal end 17 may extend to (and/or couple to) the proximal balloon shoulder 20 of the balloon 5. In which case, the proximal shaft 3 may not extend into the balloon interior 15.

The support member 8 can extend distally in parallel to, and radially offset from (not coaxial with) the inner shaft 10. For example, both the support member 8 and the inner shaft 10 can extend distally through the balloon 5, and the support member 8 can be laterally beside the inner shaft 10. The support member 8 can therefore support the inner shaft 10.

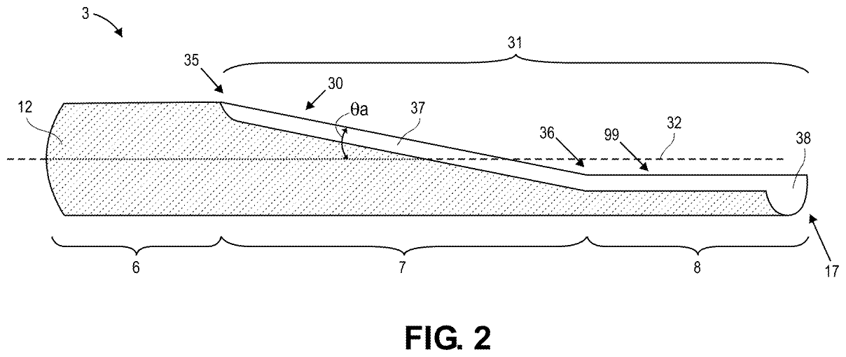

In another embodiment, the support member 8 may only extend through the balloon 5. In which case, the distal portion 7 of the proximal shaft 3 may have a distal end (e.g., end 36 as shown in FIG. 2) that is aligned, e.g., along the longitudinal axis 32, to the distal midshaft end 19, and the support member 8 may be coupled to the distal end of the distal portion 7. In another embodiment, the proximal shaft 3 may extend through the balloon 5 and out of the distal balloon shoulder 21 into the environment. In another embodiment, the distal end 17 of the proximal shaft 3 may be a floating end within the balloon interior 15. In which case, a portion of the proximal shaft 3 may be coupled to the proximal balloon shoulder 20, while the distal end 17 is disposed within the balloon interior 15 and is not coupled to (e.g., a wall of the balloon interior 15 of) the balloon 5. The proximal shaft 3 may be floating with respect to both balloon shoulders 20 and 21.

In one embodiment, the proximal shaft 3 includes a proximal lumen 12 that is in fluid communication with an interior of the adapter 2, and is in fluid communication with the midshaft lumen 13 of the midshaft 4. In some embodiments, the proximal shaft 3 may be at least partially tubular shaped and/or may be at least partially semi-tubular shaped. In particular, the outer portion 33 and the proximal portion 6 may be tubular shaped by having a circular cross-section, where both of these tubular portions may have a radius (or radial distance) that is constant, e.g., a constant diameter, from the proximal end 16 to the distal portion 7 along the longitudinal axis 32. The distal portion 7 may include an angled cut with respect to the longitudinal axis 32, where this portion the proximal shaft 3 transitions from being tubular shaped to being semi-tubular shaped by having a cross-section that is arc shaped. In one embodiment, an arc angle with respect to the longitudinal axis 32 decreases, as the angular cut extends distally towards the support member 8. As a result, the distal portion 7 may include an opening that allows the proximal lumen 12 to be in fluid communication with the midshaft lumen 13. In one embodiment, the support member 8 may have a semi-tubular shape extending from the distal portion 7 to the distal end 17. More about the shape and structure of the proximal shaft are further described with respect to FIGS. 2-5.

The inner shaft 10 extends distally from a proximal inner shaft end 34 and through the midshaft lumen 13 to a distal inner shaft end 22. In particular, the proximal end 34 of the inner shaft 10 may be coupled to a notch (or opening) 9 of the midshaft 4 that may be disposed distally from the proximal midshaft end 18 along the longitudinal axis 32, and the distal inner shaft end 22 of the inner shaft may be coupled to the balloon 5. In one embodiment, the inner shaft 10 may extend within the midshaft lumen 13 and through the balloon interior 15 to the distal inner shaft end 22 that is disposed outside of the (balloon interior 15 of the) balloon 5. In which case, the distal balloon shoulder 21 may be coupled to a portion of the inner shaft 10 that is proximal to the distal inner shaft end 22. In either case, the medical device 1 may have a distal, atraumatic tip 50 connected to the inner shaft 10 and/or the support member 8. The inner shaft also includes a lumen 14 that is in fluid communication (or is arranged to be in fluid communication) with the ambient environment via the notch 9. In one embodiment, the distal end 22 may include an opening that allows the inner lumen 14 to be in fluid communication with the ambient environment. In one embodiment, the inner shaft 10 is arranged (or designed) to receive one or more guidewires (not shown), which may be received into the inner shaft lumen 14 through the notch 9 and to (or out of) an opening at the distal inner shaft end 22.

As shown, the inner shaft 10 has a diagonal portion with respect to the longitudinal axis 32 that may at least partially track an angle of the angled cut of the distal portion 7 of the proximal shaft. For example, a portion of the inner shaft along a length of the distal portion 7 may be angled with respect to the longitudinal axis 32. The inner shaft 10 also includes two radiopaque markers 11 that are located within the balloon 5. In one embodiment, the inner shaft 10 may include more or less markers and/or may include markers that are disposed along the shaft at distinct locations, such as being within the midshaft 4.

The balloon 5 includes a proximal balloon shoulder 20 that is coupled to a distal midshaft end 19 of the midshaft 4, and includes a distal balloon shoulder end 21. In one embodiment, the distal balloon shoulder 21 may be coupled to the distal inner shaft end 22 of the inner shaft 10 and/or may be coupled to the distal end 17 of the proximal shaft 3. In particular, an inner surface of a balloon wall of the balloon 5 may be coupled to the inner shaft 10 and/or the proximal shaft via a bond, such as an adhesive or a thermal bond. In another embodiment, in addition or in lieu of being coupled to the distal ends 17 and 22, the balloon may be coupled to portions of either shaft. For example, the proximal balloon shoulder 20 and/or the distal balloon shoulder 21 may couple to an outer surface of the inner shaft 10 and/or an outer surface of the proximal shaft 3. The balloon also includes a balloon interior 15 that is in fluid communication with the midshaft lumen 13 of the midshaft 4.

As described herein, at least some of the shafts of the medical device 1 may be coupled to one another, forming water-tight fits, such that liquid (e.g., flowing through the medical device 1, as described herein) does not leak out into the environment. For example, the proximal midshaft end 18 may be coupled to a (e.g., surface of a) portion of the proximal shaft 3, and the distal midshaft end 19 may be coupled to the proximal balloon shoulder 20. In an embodiment, at least two of the shafts (or elements of the medical device) may be bonded together through a seal weld, which may be formed by applying heat to a portion of the medical device 1, and then applying an adhesive. As another example, portions of the medical device may be coupled together by bonding at least two portions using one or more adhesives.

In one embodiment, the medical device may comprise at least one material. As described herein, the portions of the proximal shaft may form one integrated unit. In which case, the proximal shaft may be formed from one material. For example, the proximal shaft may be a hypotube that is formed from an alloy (e.g., stainless steel) to provide the midshaft 4 and the balloon 5 with at least some rigidity. The increased rigidity can include increased column strength and/or decreased flexibility in bending. This rigidity may allow the device to better navigate a tortuous artery that would otherwise be difficult (or even impossible) to navigate with conventional catheter devices. Other portions of the device may comprise (other) materials. For example, the midshaft 4, the inner shaft 10, and/or the balloon 5 may be formed from a polymer or copolymer (e.g., a thermoplastic elastomer, such as polyether block amide).

In one embodiment, the balloon 5 may comprise one or more materials, which allow the balloon to expand (e.g., increasing its volume) in response to increased pressure within the balloon (e.g., due to liquid being flowed into the balloon), and/or retract in response to a decrease in pressure (e.g., due to the liquid being removed from the balloon). For example, the balloon may comprise a thermoplastic elastomer, such as polyether block amide. In some embodiments, the balloon may be arranged to have an expanded size, which may be between 0.5 mm to 4 mm, e.g., 1.5 mm, and a length that may be between 10 mm to 30 mm, e.g., 20 mm. In another embodiment, the length of the balloon may be between 10 mm to 150 mm. In one embodiment, the length of the balloon may be anatomy dependent. For example, the length of the balloon may be below a threshold length for PTAs or stenting procedures below an ankle of a patient, while the length may be above the threshold for PTAs or stenting procedures between a patient's knee and ankle.

As described herein, the medical device 1 may be arranged to perform a PTA or stenting procedure within an artery within a patient's lower extremity. In particular, the medical device 1 can be a rapid exchange (RX) balloon catheter in which a guidewire tracks through the notch 9 of the inner shaft 10 and through the balloon 5 (out of the distal inner shaft end 22) creating a tip of the device. To perform the PTA, an incision is made into (e.g., a leg of) a patient. A distal end of the guidewire is tracked through the incision to a target anatomical site. The distal end of the medical device 1 is loaded onto the guidewire, inserted through the incision, and tracked through the vasculature to the target anatomical site, e.g., a blocked artery. A user may guide (navigate) the medical device 1 through the artery, until the balloon 5 reaches the blockage site. The user may then (attach the adapter 2 to the proximal shaft 3 if not already attached, and) attach a syringe onto (e.g., a port 29 of the adapter, at a proximal end of) the adapter 2 that includes a liquid (e.g., saline and/or contrast medium). The user may force the liquid into the medical device in which the liquid flows through the proximal lumen 12 of the proximal shaft 3, into the midshaft lumen 13 of the midshaft 4, and into the balloon interior 15 of the balloon 5, inflating the balloon 5. The balloon may be deflated by removing the liquid. In one embodiment, the balloon may be inflated several times in order to open the blockage site.

In one embodiment, at least a portion of the medical device 1 may have a hydrophilic coating (top layer) that allows the medical device to traverse through an artery with minimal (or no) friction. For example, the balloon 5, and/or at least a portion of the midshaft 4 may be coated with a hydrophilic material.

FIG. 2 shows a side perspective view of the proximal shaft 3 along the longitudinal axis 32 of the balloon dilatation medical device 1, in accordance with an embodiment. As described herein, the proximal shaft 3 includes a proximal portion 6 that is a tube having a circular shape about the longitudinal axis 32. The proximal shaft 3 has a skive portion (or section) 31 that includes the proximal (first) portion 7 and the support member 8 (second or distal portion) that are both semi-tubes in which an inner lumen surface 38 defining the proximal lumen 12 is exposed to (or allowing for fluid communication of the proximal lumen with) an outside environment from the proximal lumen. In particular, the proximal portion 7 includes an angled cut 30 that extends from a proximal end 35 (of the distal portion) distally at an angle, θ₀, relative to and along the longitudinal axis 32 to a distal end 36 of the distal portion. This cut 30 creates an opening 37 that allows the proximal lumen 12 to be in fluid communication with the outside environment, as described herein. In addition, the support member 8 includes a horizontal cut from the distal end 36 of the distal portion 7 to the distal end 17 of the proximal shaft 3.

In one embodiment, the skive portion 31 may be formed by cutting (or removing) a portion of the proximal shaft along the longitudinal axis 32. For example, prior to being formed, the distal portion and the support member may be tubular shaped, having a similar radial distance from the longitudinal axis 32 as the proximal portion 6, such that the proximal shaft (e.g., along its entire length) is a circular tube. The skive portion 31 is formed by creating the angled cut 30 (e.g., using a cutting tool) to cut through the proximal shaft along a plane at θ₀ from the proximal end 35 to the distal end 36, and (e.g., subsequently) creating the horizontal cut 99 from the distal end 36 to the distal end 17. In another embodiment, the skive portion of the proximal shaft may be formed using a mold.

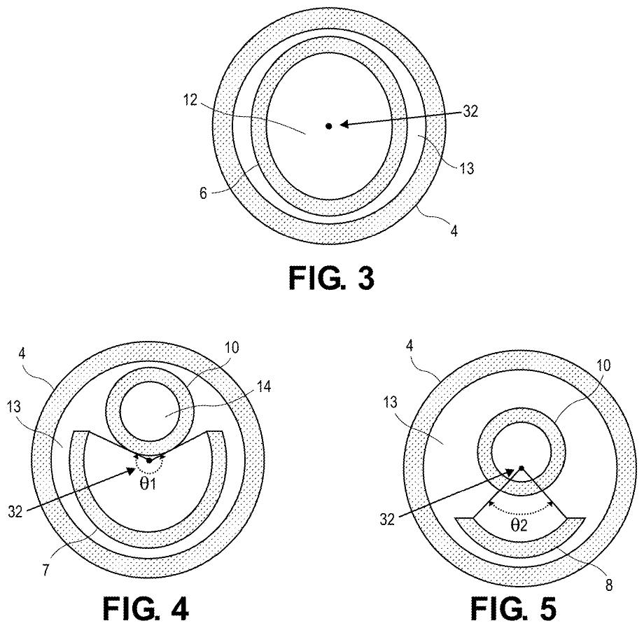

FIGS. 3-5 show cross-sectional views of the medical device 1, in accordance with an embodiment. The descriptions of these figures will be in reference to FIGS. 1 and 2. FIG. 3 shows a cross-section of the medical device 1 along the proximal portion 6 of the midshaft, as shown in FIG. 1. This cross-section shows the proximal portion 6 inside the midshaft lumen 13 of the midshaft 4, where the longitudinal axis 32 may be a center axis of one or both of the proximal portion and the midshaft. As shown, the longitudinal axis is running through the proximal lumen 12 of the proximal portion. In one embodiment, although this cross-section shows space between the (e.g., outer surface of the) proximal portion 5 and the (e.g., inner midshaft lumen surface of the) midshaft 4, both shafts may be in contact with one another. In some embodiments, the proximal portion and the midshaft may be in contact to form an interference fit, as described herein. As shown, the proximal portion and the midshaft are circular shaped. In particular, a profile of the proximal portion may be an ellipse, and a profile of the midshaft may be a circle. In one embodiment, the proximal portion may be similar shaped as the midshaft (both having a circular shape), while having a different radial distance from the longitudinal axis. In one embodiment, the cross-section shown in this figure may represent the cross-section of the medical device along a length of the proximal portion of the proximal shaft 3.

FIG. 4 shows a cross-section of the medical device 1 along the distal portion 7 of the proximal shaft. In particular, this cross-section includes the distal portion 7 of the proximal shaft 3 and the inner shaft 10 that are within the midshaft lumen 13 of the midshaft 4. Since this cross-section is along the angled cut 30 of the skive portion 31 of the proximal shaft 3, the distal portion is arc shaped that opens towards the inner shaft 10. In one embodiment, at this point along the skive portion, the distal portion has an arc angle of θ₁ from the longitudinal axis 32. In another embodiment, θ₁ may be with respect to another axis that runs parallel with the longitudinal axis 32 (which may be the case when the proximal portion is not centered with the longitudinal axis 32).

In one embodiment, the arc angle of the distal portion may change along the angled cut 30. For example, referring to FIG. 2, the distal portion may have a first arc angle at (or distal to) the proximal end 35 and may have a second, different arc angle at (or proximal to) the distal end 36. In one embodiment, the arc angle may decrease along (at least a portion of) a length of the distal portion from the proximal end 35 to the distal end 35. Thus, the first arc angle may be greater than the second arc angle, described herein. In one embodiment, the change in the arc angle may be due to the proximal shaft having a circular shape and that the skive portion 31 is formed by an angled cut 30 along θ₀, as described herein.

FIG. 5 shows another cross-section of the medical device 1 along the support member 8. Specifically, this figure shows the support member 8 and the inner shaft 10 disposed within the midshaft lumen 13 of the midshaft 4. The support member 8, as described herein, can be semi-tubular shaped, being an arc with an arc angle θ₂. In one embodiment, θ₁ is greater than θ₂. In some embodiments, θ₂ may be the arc angle of at least a portion of the support member 8 along the longitudinal axis 32. In another embodiment, θ₂ may be the arc angle of the entire support member 8, as shown in FIG. 2. In one embodiment, θ₂ may be the arc angle at the distal end 36 of the distal portion 7. In which case, the support member may have an arc angle of θ₂ along at least a portion of the member (along the longitudinal axis 32) from the distal end 36 of the distal portion 7.

Also shown in this figure, the inner shaft 10 has moved from its position shown in FIG. 4 towards the support member 8. In one embodiment, the inner shaft may move downward along a length of the medical device that corresponds to the distal portion 7 of the proximal shaft 3. For example, referring to FIG. 2, the inner shaft 10 may run along the distal portion 7 at an angle with respect to the longitudinal axis 32. In one embodiment, the angle along which the inner shaft runs may be the same as θ₀. Distal from the distal portion, however, the inner shaft may run parallel to the longitudinal axis, as shown in FIG. 1.

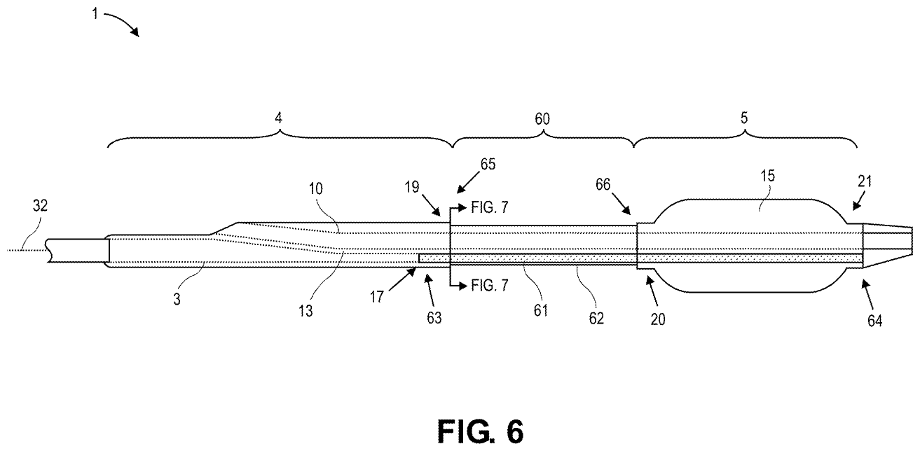

FIG. 6 shows a side view of the medical device 1, in accordance with another embodiment. The device 1 includes a distal (or outer) shaft 60 that is at least partially disposed between the midshaft 4 and the balloon 5. Thus, the midshaft 4 may couple to the balloon 5 via the distal shaft 60. For example, the distal shaft 60 includes a proximal end 65 that is attached to the distal midshaft end 19, and includes a distal end 66 that is attached to the proximal balloon shoulder 20 of the balloon 5. In one embodiment, the distal shaft 60 may be coupled at either or both ends via an adhesive or thermal joint or bond. Similarly, the proximal balloon shoulder 20 may be sized to receive the distal end 66, and thereby may create a water-tight fit.

In one embodiment, the distal shaft 60 may include (or be formed from) one or more materials. In some embodiments, the distal shaft may include a different material than that of the proximal shaft 3 and/or the midshaft 4. For instance, the proximal shaft 3 may be a hypotube formed from an alloy, as described herein, whereas the distal shaft may be formed from a polymer or copolymer. This may allow the distal shaft 60 to be flexible (or more flexible than the proximal shaft 3). In one embodiment, the distal shaft may have a length along the longitudinal axis 32 that is greater than a length of the midshaft 4. In another embodiment, the midshaft 4 may be longer (along the axis 32) than the distal shaft 60.

This figure also illustrates that the device includes a support wire 61 that extends distally through the distal shaft 60 and the balloon 5, and includes a distal wire end 64 that is coupled to the distal balloon shoulder 21. In particular, the support wire 61 may be a part of the proximal shaft 3. In particular, a proximal end 63 of the support wire 61 may be coupled (or attached) to the distal end 17 of the proximal shaft 3. In one embodiment, the support wire may be coupled via a thermal bond or an adhesive bond. In some embodiments, the support wire 61 may be of a same material as the proximal shaft 3, or may be of a different (or include a different) material from the proximal shaft 3. In one embodiment, the support wire 61 may have a different shape and/or size than at least a portion of the proximal shaft 3. As described herein, the distal end 17 of the proximal shaft 3 may be semi-tubular shaped (e.g., have an arc cross-section), while the support wire 61 may be tubular shaped (e.g., having an annular cross-section) or circular shaped (e.g., a solid circular wire). In another embodiment, the support wire may be semi-tubular shaped.

In one embodiment, the support member 8 of the proximal shaft 3 may be the support wire 61. For example, referring to FIG. 2, the support wire 61 may be coupled to the proximal shaft at (or near) the distal end 36 of the distal portion 7, when the support wire is attached to the shaft. In another embodiment, the proximal shaft 3 may include the support wire 61 in addition to having (at least a portion of) the support member 8. In the case in which the support wire 61 is coupled to the support member 8 of the proximal shaft 3, a length of the support member may be less than a length of the support member when the proximal shaft does not include the support wire 61. For instance, the support member may be entirely disposed within the midshaft 4, as opposed to extending out of the midshaft and into the balloon.

Returning to the distal shaft 60, this shaft includes a shaft lumen 62 that is in fluid communication with the midshaft lumen 13 and the balloon interior 15. As a result, the inner shaft 10 and/or the support wire 61 extend through the shaft lumen 62 from the midshaft lumen 13 of the midshaft 4 and into the balloon interior of the balloon 5, as shown.

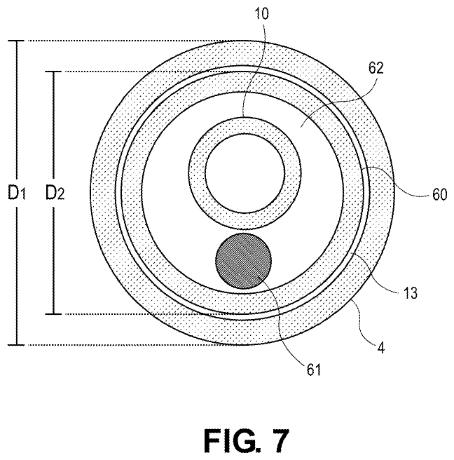

FIG. 7 shows another cross-sectional view of the medical device 1, in accordance with another embodiment. In particular, this figure is showing a cross section at the proximal end 65 (and/or the distal midshaft end 19), as shown in FIG. 6. This figure is showing that the distal shaft 60 is inside the midshaft lumen 13 of the midshaft 4, at or near the distal midshaft end 19. Inside the shaft lumen 62 of the distal shaft 60 is the inner shaft 10 and the support wire 61. In one embodiment, the shaft lumen 62 may at least partially include the semi-tubular shaped support member 8 of the proximal shaft 3, as shown in FIGS. 1-5, when the proximal shaft includes the support member.

As shown, the distal shaft 60 is circular shaped and has a diameter of D₂, while the midshaft 4 has a diameter of D₁, which is greater than D₂. In one embodiment, D₁ may be greater than D₂ such that the distal shaft 60 may be fitted into the midshaft and attached to the midshaft through an interference fit, as described herein.

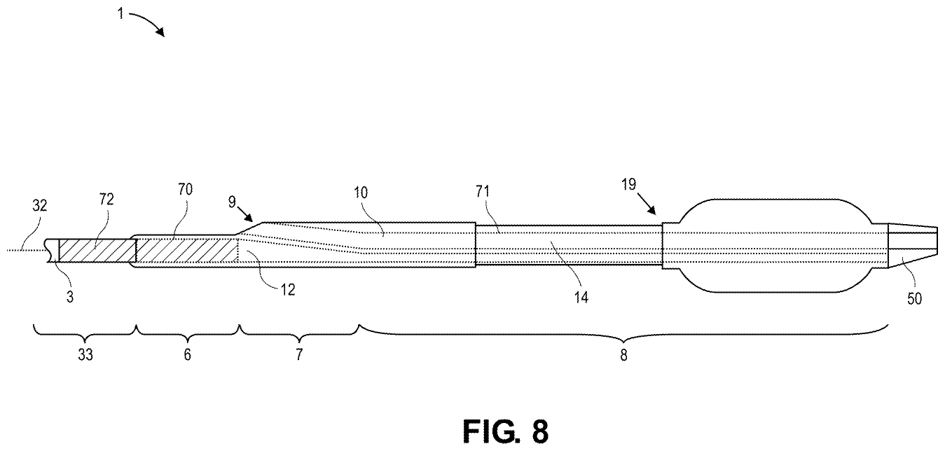

FIG. 8 shows a side view of the medical device 1, in accordance with another embodiment. This figure is showing that portions (or at least one portion) of the medical device 1 may include one or more cut patterns. In one embodiment, a cut pattern is a pattern of one or more cuts into and/or through a wall of an object such that an inner surface of the wall may be in fluid communication with an outer surface of the wall, through a cut. In some embodiments, a cut may remove one or more portions of the wall and/or separate two or more portions of the wall from each other. In one embodiment, each cut may form one or more (hollow) shapes in the wall of the object. More about cut patterns is described herein. In one embodiment, cut patterns in a shaft may allow the shaft to be flexible, while still maintaining a sufficient amount of rigidity.

Turning now to FIG. 8, the proximal shaft 3 includes a proximal shaft wall 70 that may at least partially surround the proximal lumen 12 along at least a portion of (a length of the proximal shaft along) the longitudinal axis 32. As shown, at least a portion of the proximal shaft wall 70 of the proximal shaft 3 includes a (first) cut pattern 72 that extends (or is a part of the shaft wall 70) along the longitudinal axis 32. In particular, referring to FIGS. 1 and 2, the cut pattern 72 extends proximally from (e.g., the proximal end 35 of) the distal portion 7 to the outer portion 33 of the proximal shaft 3. In one embodiment, the cut pattern 72 may extend proximally from the distal portion 6 to the proximal end 16 of the proximal shaft 3. In another embodiment, the proximal portion 6 of the proximal shaft 6 may be the only portion of the proximal shaft that includes the cut pattern 72. In which case, the proximal shaft wall 70 of the proximal portion 6 may include one or more (first) cuts in the first cut pattern, whereas the proximal shaft wall 70 of the distal portion 7 (and/or the support member 8) may not include cuts, thereby having a solid proximal shaft wall 70. In one embodiment, the proximal shaft 3 (and/or one or more other portions of the medical device 1) may include one or more cut patterns, which may extend along one or more portions of the device.

In addition, the inner shaft 10 includes an inner shaft wall 71, which may be a solid wall that at least partially surrounds a center longitudinal axis (e.g., axis 32) of the shaft 10 that extends at least a length of the shaft. As shown, the inner shaft extends distally from the notch 9 of the midshaft to (and/or beyond) the tip 50 of the device 1.

As described herein, the medical device may include one or more cut patterns. In one embodiment, when the device includes multiple cut patterns, each may be the same type of cut pattern, or each may be different from one another. In another embodiment, one or more shafts of the medical device 1 may include one or more distinct types of cut patterns. For example, the portion 6 of the proximal shaft 3 may include the cut pattern 72, while another portion of the proximal shaft, such as portion 33 may include one or more other cut patterns. As another example, one or more cut patterns may span along a same portion of the inner shaft.

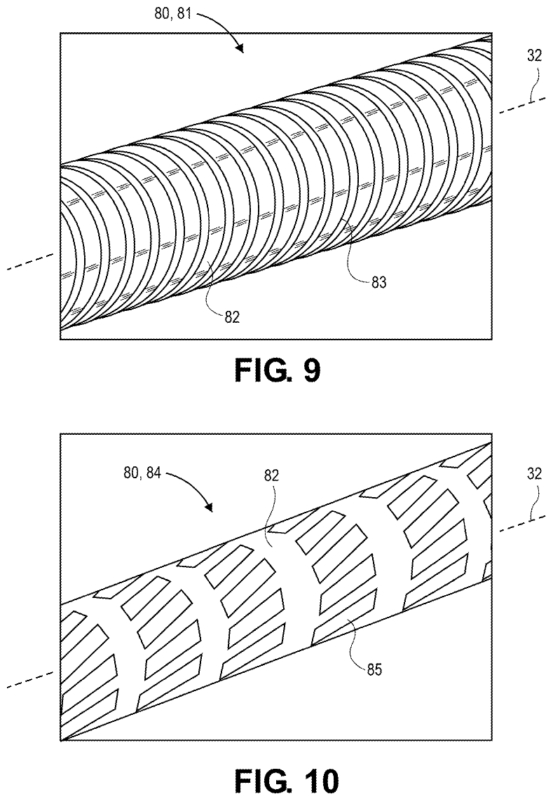

FIGS. 9 and 10 each show a different cut pattern of a shaft 80 according to an embodiment. In one embodiment, the shaft 80 may include the proximal shaft 3, the midshaft 4, the inner shaft 10, and/or the distal shaft 60. Turning to FIG. 9, this figure shows that the shaft 80 has a continuous cut pattern 81. In particular, a wall 82 of the shaft 80 includes one continuous cut 83, e.g., a spiral cut, around the longitudinal axis 32 of the shaft 80. As shown, the cut 83 may pierce the wall, such that the cut makes an opening through the wall. In one embodiment, the spiral cut pattern may improve both deliverability and pushability of a catheter delivery system when implemented into the medical device 1. Such a spiral cut may be utilized in one or more locations of the medical device (e.g., as shown in FIG. 8) to improve flexibility while use during a PTA. Turning to FIG. 10, this figure shows that the shaft has a discontinuous cut pattern 84. For example, the wall 82 of the shaft includes several rows of elongated cuts that are positioned on the shaft and along the longitudinal axis 32, where each row includes one or more cuts that are positioned around the shaft and about the longitudinal axis. The one or more cuts can be separated from each other by wall segments such that the cuts are discontinuous with each other. In one embodiment, the cut pattern may provide similar benefits to the medical device as described with respect to the spiral cut pattern.

In one embodiment, other cut patterns are possible. For example, the shaft may include a square cut patterns, where one or more rows of one or more square shaped cuts are formed on the shaft and along the longitudinal axis 32, and as describe herein, each row of one or more square shaped cuts may be positioned around the shaft. As another example, the shaft may include a triangular cut pattern, where triangular-shaped cuts are formed in the wall 82. The discontinuous cut pattern 84 could include several I-beam shaped cuts that are interleaved with each other. In another embodiment, any shaped pattern may be formed within the wall.

As described herein, the medical device 1 may include one or more cut patterns. For example, turning to FIG. 8, the support member 8 may include one or more cut patterns, where the proximal wall 70 along the support member may include one or more cuts that each form one or more openings in the member, thereby creating the cut pattern. In one embodiment, one or more shafts with one or more cut patterns may include layers that prevent (or reduce) fluid communication from inside a shaft to outside the shaft. For example, the proximal shaft 3 may include an outer layer that is formed on an outside surface of the shaft in order to prevent (or reduce) fluid communication between the proximal shaft lumen 12 and the midshaft lumen 13, along the proximal portion 6 of the proximal shaft 3. In one embodiment, this outer layer may include one or more materials, such as a polymer or copolymer. In another embodiment, in addition to or in lieu of having the outer layer, the shaft may include an inner layer that is formed on an inner surface of the proximal shaft 3.

As described thus far, the medical device may be a RX balloon catheter. In another embodiment, the medical device may be an over-the-wire (OTW) balloon catheter in which a guidewire is navigated through the entire length of the guidewire.

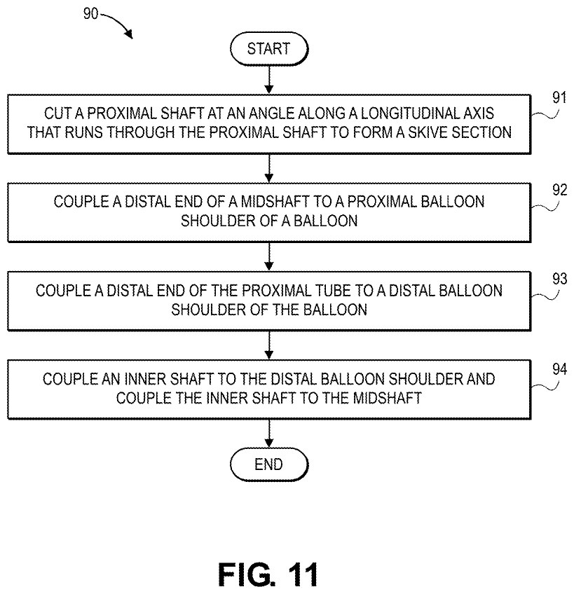

FIG. 11 is a flowchart of one embodiment of a process 90 for manufacturing a balloon dilatation medical device 1. The description of this process 90 is in reference to FIGS. 1 and 2. In one embodiment, the process 90 may be performed by a single (e.g., manufacturing) machine or multiple machines to manufacture one or more medical devices 1, as illustrated herein. In some embodiments, at least some of the operations within this process may be automatically performed by one or more machines. For example, at least some of the operations may be performed without user intervention. In another embodiment, at least some of the operations may be manually performed by one or more users operating one or more machines.

The process 90 begins by cutting a proximal shaft at an angle along a longitudinal axis that runs through the proximal shaft to form a skive section that has an arc-shaped cross section (at block 91). In particular, the skive section 31 includes the distal portion 7 of the proximal shaft 3, where the distal portion is formed by cutting into the proximal shaft starting at the proximal end 35 at an angle, θ₀, to create the angled cut 30, which may terminate at the distal end 36 of the distal portion 7. In one embodiment, the skive section 31 may also include (at least a portion of) the support member 8 that may have an arc-shaped cross-section that may be formed by a horizontal (or substantially horizontal) cut 99 from the distal end 36 of the angled cut 30 to the distal (terminating) end 17 of the proximal shaft 3, as shown in FIG. 2. In another embodiment, the horizontal cut may terminate before the distal end 17. In some embodiments, the proximal shaft may be cut using one or more cutting tools. In which case, the proximal shaft 3 may be a tube extending from its proximal end to its distal end, and to form the skive portion, the cutting tool may cut (e.g., laterally) into the proximal shaft to remove a portion of the shaft. As a result, a portion of the proximal shaft that is proximal to the skive portion may be tubular shaped (e.g., having a circular cross-section), while the skive portion may be semi-tubular shaped, as described herein. In another embodiment, the skive portion 31 of the proximal shaft may be formed using one or more molds.

The process 90 couples a distal end of a midshaft to a proximal balloon shoulder of a balloon (at block 92). For example, the distal midshaft end 19 of the midshaft 4 may be attached to the proximal balloon shoulder 20. Both may be attached using a thermal bond, an adhesive bond, and/or an interference fit. As a result, the midshaft lumen 13 may be in fluid communication with the balloon interior 15 of the balloon 5.

The process 90 couples a distal end of the proximal shaft to a distal balloon shoulder of the balloon (at block 93). In particular, the distal end 17 of the support member 8 may be attached to (e.g., an interior surface of) the balloon interior 15. The distal end may be attached through an adhesive bond, for example. In one embodiment, at least a portion of the proximal shaft may be disposed within the midshaft, such that the skive section may be at least disposed within the midshaft lumen 13 of the midshaft 4.

The process 90 couples an inner shaft to the distal balloon shoulder of the balloon and couples the inner shaft to the midshaft (at block 94) Specifically, the distal inner shaft end 22 may be coupled to the distal balloon shoulder 21, while a proximal inner shaft end 34 may be coupled to the notch 9 of the midshaft 4.

Some embodiments may perform variations to at least one of the process 90 described herein. For example, the specific operations of a process may not be performed in the exact order shown and described. The specific operations may not be performed in one continuous series of operations and different specific operations may be performed in different embodiments. For example, the process 90 may include operations to form one or more cut patterns on the medical device 1. As an example, the process 90 may include forming one or more first cuts (e.g., a continuous cut) in a first cut pattern on a portion (e.g., the proximal portion 6) of the proximal shaft 3, as shown in FIG. 10. The process 90 may include forming one or more second cuts (e.g., discontinuous cuts) in a second cut pattern on another portion of the proximal shaft and/or on a portion of another shaft of the catheter. In one embodiment, the cuts may be formed using one or more cutting tools that cut into (e.g., a shaft of) the medical device. In which case, the tool may remove one or more portions of a shaft in order to form cuts as openings into the shaft.

In another embodiment, the midshaft may be coupled to the balloon via one or more other shafts. For example, the process 90 may couple the distal end of the midshaft to the proximal balloon shoulder of the balloon by attaching a proximal end of a distal shaft (e.g., shaft 60 shown in FIG. 6) to the distal end of the midshaft and attaching a distal end of the distal shaft to the proximal balloon shoulder. In one embodiment, the distal shaft may be of a different (or same) material as the midshaft. For instance, the distal shaft may be more formed from one or more materials that allow the distal shaft to be more flexible than the midshaft.

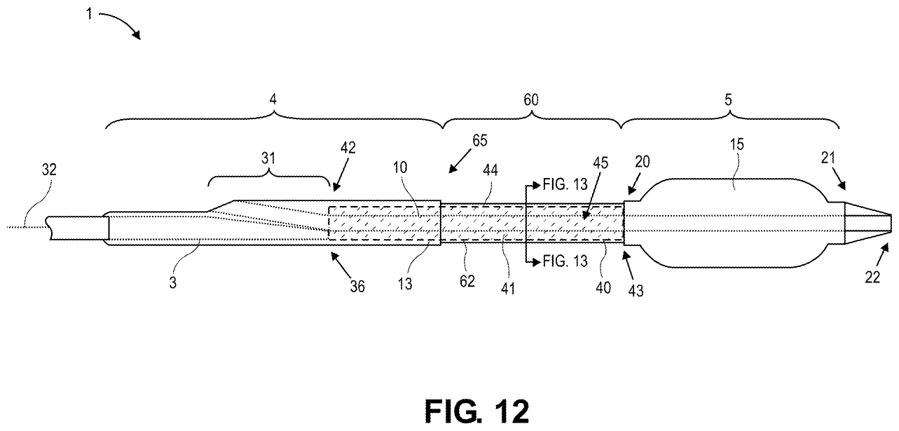

FIG. 12 shows a side view of the medical device 1, in accordance with an embodiment. The device includes one or more elements as described herein, such as a proximal shaft 3, a midshaft 4 that is coupled to the proximal shaft and includes a midshaft lumen 13, a balloon 5 that includes a balloon interior 15 that is in fluid communication with the midshaft lumen 13, where the balloon includes a proximal balloon shoulder 20 and a distal balloon shoulder, and an inner shaft 10 that is extending distally through the midshaft lumen 13 from a proximal inner shaft end 34 to a distal inner shaft end 22 that is coupled to the distal balloon shoulder 21. The device 1 also includes an intermediate shaft 40 that extends distally through a portion of the midshaft lumen 13 of the midshaft 4, and the distal lumen 62 of the distal shaft 60, along the longitudinal axis 32. In particular, the intermediate shaft 40 includes a proximal end 42 that is coupled to the proximal shaft 3, and includes a distal end 43 that is coupled to the proximal balloon shoulder 20 of the balloon 5. In particular, the intermediate shaft is coupled to the end of the angled portion of the skive section 31 of the proximal shaft. For example, the skive section terminates at the proximal end 42 of the intermediate shaft, to which the distal end 36 of the skive section is attached. In one embodiment, the intermediate shaft may be a (or a part of a) support member that couples the midshaft 4 to the balloon 5. In another embodiment, the intermediate shaft 40 may extend through the balloon interior 15 and couple to the distal balloon shoulder 21 of the balloon.

In one embodiment, the intermediate shaft 40 may also include an intermediate shaft wall 44 that at least partially surrounds an intermediate lumen 41 that is in fluid communication with (at least) the midshaft lumen 13 and/or the balloon interior 15. As shown, the inner shaft 10 may at least partially extend through the intermediate lumen 41 of the intermediate shaft. In particular, a portion of the inner shaft 10 extends into the intermediate shaft at the proximal end 42 and out of the distal end 43 (e.g., along the longitudinal axis 32). This figure also shows that the intermediate shaft wall 44 of the intermediate shaft includes a (e.g., second) cut pattern 45, where the pattern extends from the proximal end 42 to the distal end 43 of the intermediate shaft. In one embodiment, the cut pattern 45 may partially extend along the wall 33 of the intermediate shaft.

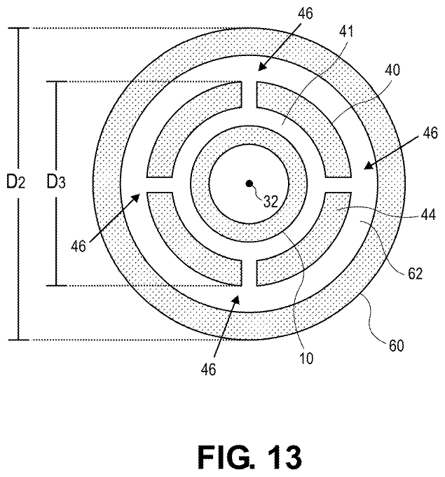

FIG. 13 shows another cross-sectional view of the medical device 1, in accordance with an embodiment. In particular, this figure is showing a cross-section of the medical device along the distal shaft 60, as shown in FIG. 12. This figure is showing that the inner shaft 40 is inside the shaft lumen 62 of the distal shaft 60, and the inner shaft 10 is inside the lumen 41 of the intermediate shaft 40. In one embodiment, the longitudinal axis 32 may be a center longitudinal axis through which the distal shaft 60, the intermediate shaft 40, and/or the inner shaft 10 extend along. As shown, the intermediate shaft 40 may have a cross-section having a circular shape, and has a diameter of D₃, which may be less than D₂, but greater than a diameter of the inner shaft 10, such that the inner shaft may extend through the intermediate shaft 40.

This figure also shows a cross-sectional view of the cut pattern 45 of the intermediate shaft 40. Specifically, this figure shows that the intermediate shaft wall 44 includes several cuts (or openings) 46 that allow the intermediate shaft lumen 41 to be at least partially in fluid communication with the lumen 62 of the distal shaft 60. In one embodiment, the wall 44 may include more or less openings, where the openings may be arranged differently.

While certain embodiments have been described and shown in the accompanying drawings, it is to be understood that such embodiments are merely illustrative of and not restrictive on the broad invention, and the invention is not limited to the specific constructions and arrangements shown and described, since various other modifications may occur to those of ordinary skill in the art. The description is thus to be regarded as illustrative instead of limiting.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What is claimed is:

1. A catheter, comprising:
a midshaft having a distal midshaft end;
an inner shaft extending distally through the midshaft to a distal inner shaft end;
a balloon having a proximal balloon shoulder coupled to the distal midshaft end and a distal balloon shoulder coupled to the inner shaft; and
a proximal shaft including a support member that extends through the balloon at least to the distal balloon shoulder.

2. The catheter of claim 1, wherein the midshaft comprises a midshaft lumen, wherein the proximal shaft includes a proximal portion that extends proximally from a proximal midshaft end, and a distal portion that is coupled to the support member that extends distally into the midshaft lumen, and wherein the distal portion includes an angled cut that extends distally at an angle relative to a longitudinal axis that runs through the proximal shaft.

3. The catheter of claim 2, wherein the proximal portion is circular shaped, and the distal portion is arc shaped, wherein a first arc angle at a proximal end of the distal portion is greater than a second arc angle at a distal end of the distal portion, wherein the support member is attached to the distal end of the distal portion and is arc shaped having the second arc angle along at least a portion of the support member from the distal end of the distal portion.

4. The catheter of claim 3, wherein the proximal portion, the distal portion, and the support member are one unit.

5. The catheter of claim 2, wherein the support member is a wire.

6. The catheter of claim 2, wherein the proximal portion includes a proximal shaft wall having a plurality of cuts in a cut pattern.

7. The catheter of claim 6, wherein the cut pattern includes one or more of a continuous cut pattern or a discontinuous cut pattern.

8. The catheter of claim 1, wherein the distal midshaft end is attached to the proximal balloon shoulder, wherein the midshaft includes a midshaft lumen, the proximal shaft includes a proximal lumen, and the balloon includes a balloon interior, wherein the proximal lumen is in fluid communication with the midshaft lumen, which is in fluid communication with the balloon interior, and wherein the support member extends distally within the midshaft lumen and within the balloon interior to a distal end at or distal to the distal balloon shoulder.

9. The catheter of claim 1 further comprising a distal shaft having a proximal outer end that is attached to the distal midshaft end and a distal outer end that is attached to the proximal balloon shoulder, wherein the proximal shaft comprises a first material and the distal shaft comprises a second, different material.

10. A catheter, comprising:
a proximal shaft;
a midshaft coupled to the proximal shaft and including a midshaft lumen;
a balloon including a balloon interior that is in fluid communication with the midshaft lumen, wherein the balloon includes a proximal balloon shoulder and a distal balloon shoulder;
an inner shaft extending distally through the midshaft lumen from a proximal inner shaft end to a distal inner shaft end, wherein the inner shaft is coupled to the distal balloon shoulder; and
an intermediate shaft including a shaft wall that at least partially surrounds an intermediate shaft lumen through which the midshaft lumen fluidly communicates with the balloon interior, wherein the intermediate shaft has a plurality of cuts in a cut pattern, and is coupled to the proximal shaft and to the proximal balloon shoulder of the balloon, wherein at least a portion of the inner shaft extends through the intermediate shaft lumen.

11. The catheter of claim 10, wherein the midshaft comprises a distal midshaft end, wherein the catheter further comprises a distal shaft having a proximal outer end that is coupled to the distal midshaft end and a distal outer end that is coupled to the proximal balloon shoulder, wherein the distal shaft includes a distal shaft lumen through which the intermediate shaft extends through.

12. The catheter of claim 11, wherein the distal shaft has a smaller diameter than a diameter of the midshaft at the midshaft distal end.

13. The catheter of claim 10, wherein the proximal shaft comprises a first portion that is coupled to an adapter and a second portion that is at least partially housed within the midshaft lumen, wherein the second portion includes a skive section that is arch-shaped and has a decreasing arc angle with respect to a longitudinal axis that terminates at a proximal end of the intermediate shaft.

14. The catheter of claim 13, wherein the plurality of cuts is a plurality of first cuts and the cut pattern is a first cut pattern, wherein the proximal shaft includes a proximal shaft wall that has a plurality of second cuts in a second cut pattern along at least a portion of the first portion.

15. The catheter of claim 14, wherein the first cut pattern and the second cut pattern include at least one of a continuous cut pattern or a discontinuous cut pattern.

16. The catheter of claim 14, wherein the second cut pattern is proximal from a distal end of the skive section along the longitudinal axis.

17. A method of manufacturing a catheter, the method comprising:
cutting a proximal shaft at an angle along a longitudinal axis that runs through the proximal shaft to form a skive section that has an arc-shaped cross section;
coupling a distal midshaft end of a midshaft to a proximal balloon shoulder of a balloon, wherein a midshaft lumen is in fluid communication with a balloon interior of the balloon;
coupling a distal shaft end of the proximal shaft to a distal balloon shoulder of the balloon, wherein the skive section is at least disposed within the midshaft lumen of the midshaft; and
coupling an inner shaft to the distal balloon shoulder and coupling the inner shaft to the midshaft.

18. The method of claim 17, wherein an inner proximal end of the inner shaft is coupled to a notch of the midshaft that is disposed distally from a proximal end of the midshaft along the longitudinal axis, and the inner shaft extends within the midshaft lumen and through the balloon interior to an inner distal end of the inner shaft that is disposed outside the balloon interior.

19. The method of claim 17 further comprising forming one or more cuts in a cut pattern on at least a portion of the proximal shaft.

20. The method of claim 19, wherein the cut pattern includes one or more of a continuous cut pattern or a discontinuous cut pattern.

21. The method of claim 17, wherein coupling the distal midshaft end of the midshaft to the proximal balloon shoulder of the balloon comprises attaching a proximal end of a distal shaft to the distal midshaft end of the midshaft and attaching a distal shaft end of the distal shaft to the proximal balloon shoulder.

22. The method of claim 21, wherein a lumen of the distal shaft is in fluid communication with the midshaft lumen and the balloon interior such that the inner shaft extends within the midshaft lumen, the lumen of the distal shaft, and the balloon interior, and wherein the distal shaft is a different material than the midshaft.
